(19) United States
(12) Patent Application Publication
(10) Pub. No.: US 2022/0013220 A1
(43) Pub. Date: Jan. 13, 2022

(54) DOCTRAX REFERRAL OPTIMIZATION SOLUTION

(71) Applicant: Precision Medical Partners, LLC, Sandy Hook, CT (US)

(72) Inventor: RORY DOYL, SR., SANDY HOOK, CT (US)

(73) Assignee: Precision Medical Partners, LLC, Sandy Hook, CT (US)

(21) Appl. No.: 17/369,809

(22) Filed: Jul. 7, 2021

Related U.S. Application Data
(60) Provisional application No. 63/049,007, filed on Jul. 7, 2020.

(51) Int. Cl. G16H 40/20; G06Q 40/08; H04L 29/06; G06F 3/0482; H04L 29/08

(52) U.S. Cl. CPC G16H 40/20; G06Q 40/08; H04L 63/083; G06Q 20/127; H04L 67/306; H04L 67/12; G06F 3/0482

(57) ABSTRACT

A system for referral tracking and growth platform is disclosed for private healthcare providers. The system captures real-time, specific, referral-tracking data in a manner to increase connectivity between physicians and optimize patient experience.

### PRIORITY CLAIM

This application claims the benefit of priority from provisional application Ser. No. 63/049,007, which this application incorporates by reference.

### BACKGROUND OF THE INVENTION

### Technical Field

This invention relates to medical services referral optimization. In particular, the invention relates to a system for allowing medical services providers to optimize referrals between each other.

### Related Art

Medical referral systems are known in the art. Doctors, specialists and other physicians communicate, either directly, via phone, email or via insurance agencies, patient information for referrals for specific medical conditions.

However, the known systems are inefficient due to complicated information entry, communication and cross-reference information. In the art, patients are often referred to other providers who do not participate in their insurance plans, or where the provider may no longer be in-network with a particular commercial provider and understanding if the patient has out-of-network coverage which will increase both provider and patient satisfaction. Patients often waste time or schedule appointments and the provider is not interested in pursuing treatment because the provider is unable to be paid.

Therefore, a need exists for an efficient referral system to optimize patient treatment between different medical treatment services.

### SUMMARY

The invention discloses a system for referral tracking and growth platform for private healthcare providers. The system captures real-time, specific, referral-tracking data in a precise and simplistic manner to increase connectivity between physicians and optimize patient experience. The system allows referral tracking data across the clinical spectrum, including, but not limited to primary care, internal medicine, family practice; endocrinology, rheumatology, pediatrics, chiropractics, physical therapy; physiatry, interventional pain, anesthesiology, cardiology, neurology, urgent care, behavioral health, dermatology, concierge medicine, medispas, ophthalmology, general surgery, orthopedics, bariatrics, vascular, plastic surgery, cardiac, neurosurgery, radiology; imaging centers, nutritionists, dentists, oral surgeons, cannabidiol (CBD), hemp dispensing and other medical services that one of skill in the art would understand.

The system provides clarity for both referring providers and patients regarding the providers' insurance status. This may include Medicare, Medicaid, Commercial (both in and out of network status), private pay, worker's compensation or other medical care provider insurance or payment plans.

The system will serve to avoid referrals between healthcare providers where the insurance make-up of the patient is not a viable option for the provider receiving the referral.

The provider who is sending the referral will have access to their networks' specialty, location and insurance make-up and will understand the appropriate provider based on both the patient's clinical need and insurance coverage.

Preferably, providers all pay a flat fee for the mobile subscription service and another flat fee for the web-based version. Both versions offer a marketing automation platform which allows providers to disseminate information quickly to other health care providers regarding new services, locations, rental space and educate patients about new services as well.

The invention allows that medical care can be solely determined by patients and doctors, and provides for selection over member physicians medical decisions or referral patterns.

Other systems, methods, features and advantages of the invention will be, or will become, apparent to one with skill in the art upon examination of the following figures and detailed description. It is intended that all such additional systems, methods, features and advantages be included within this description, be within the scope of the invention, and be protected by the following claims.

### BRIEF DESCRIPTION OF THE DRAWINGS

The invention can be better understood with reference to the following drawings and description. The components in the figures are not necessarily to scale, emphasis instead being placed upon illustrating the principles of the invention. Moreover, in the figures, like referenced numerals designate corresponding parts throughout the different views.

FIG. 1 illustrates an example system for communication between medical services providers and other providers to implement the disclosure.

FIG. 2 illustrates an example flowchart for the system and method described in the disclosure.

FIG. 3 illustrates an example graphical user interface described in the disclosure.

FIG. 4 illustrates another example graphical user interface described in the disclosure.

FIG. 5 illustrates another example graphical user interface described in the disclosure.

FIG. 6 illustrates another example graphical user interface described in the disclosure.

### DETAILED DESCRIPTION OF THE PREFERRED EMBODIMENTS

The elements in the Figures interoperate as explained in more detail below. Before setting forth the detail explanation, however, it is noted that all of the discussion below, regardless of the particular implementation being described, is exemplary in nature, rather than limiting. For example, although selected aspects, features, or components of the implementations are depicted as being stored in memories, all or part of systems and methods consistent with the display systems may be stored on, distributed across, or read from other machine-readable media, for example, secondary storage devices such as hard disks, floppy disks, and CD-ROMs; a signal received from a network, including a cloud computing network or other remote network; or other forms of ROM or RAM either currently known or later developed.

Although specific components of the architecture will be described, methods, systems, and articles of manufacture consistent with the architecture may include additional or different components. For example, a processor may be implemented as a microprocessor, microcontroller, application specific integrated circuit (ASIC), discrete logic, or a combination of other type of circuits or logic. Similarly, memory, may be DRAM, SRAM, Flash, or any other type of memory. Flags, data, databases, tables, and other data structures may be separately stored and managed, may be incorporated into a single memory or database, may be distributed, or may be logically and physically organized in many different ways. Programs may be parts of a single program, separate programs, or distributed across several memories and processors.

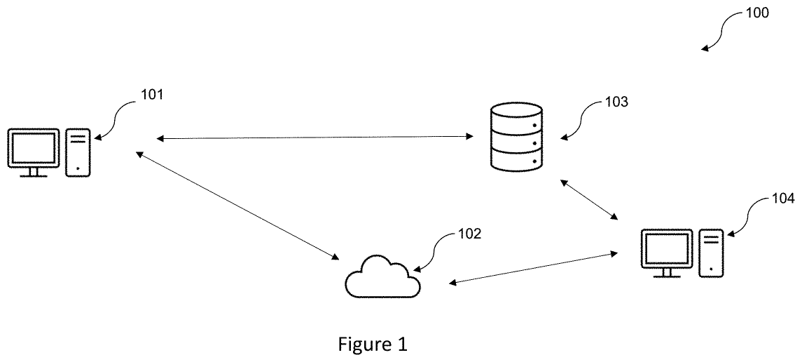

FIG. 1 illustrates an embodiment of the disclosure that enables medical referral optimization system (100). Devices may be connected across a network as shown in FIG. 1 (100), to allow communication between a medical services provider and a targeted medical services provider for referral services. Examples of network devices in the network (101) include, but are not limited to, computing devices (101, 104), servers (103), cloud computing networks (102) or other networked devices known to know of skill in the art. Memory and other storage devices that are adapted to implement elements of the disclosed invention are included in these devices (101-104) as known to one of skill in the art.

In one embodiment of the disclosure, a system for medical referral optimization is disclosed. The system includes a processor, a memory device coupled to the processor, and software operable on the processor and memory device. These elements may be embodied as depicted in elements 101-104 of FIG. 1. Elements 101-104 depict representative computing and communication devices such as desktop computers, laptops, notebooks, smartphones, tablets, mobile devices, smart watches or other devices connected to a network as known to one of skill in the art.

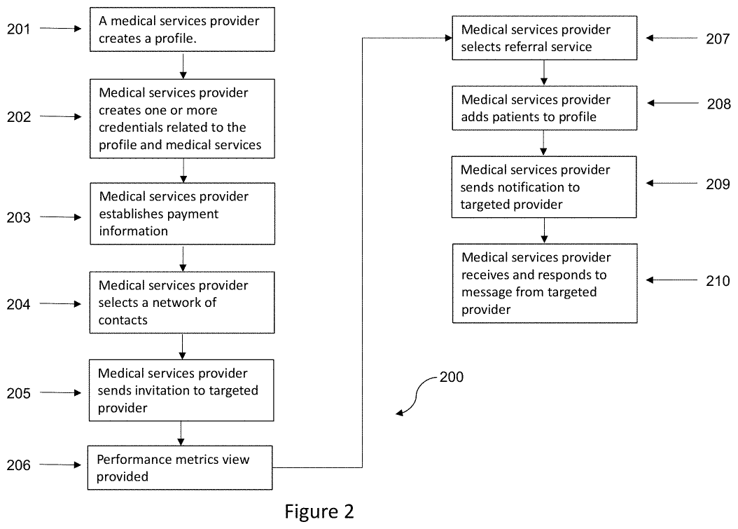

FIG. 2 illustrates an example system that implements the disclosure. The system may use the elements of FIG. 1, including hardware elements disclosed in FIG. 1 that are adapted to perform the disclosure. The software is operable to perform acts to enable the embodiment. The software is operable to allow a medical services provider to create a profile (act 201). The medical services provider may create one or more credentials related to the profile and medical services (act 202). The profile may include name of the patient, email associated with the provider, photos for the profile, phone numbers associated with the provider, specialty associated with the provider, insurance information associated with the profile, location associated with the profile and administrator emails associated with the profile to facilitate the process.

The system allows a login process and graphical user interface to allow a login to the system, including username, email, and password. Other credentials may be possible. Other authentication, such as two factor or biometric authentication may be possible.

The disclosure allows the medical services provider to establish payment information, such as name, credit card or other possible payment information to facilitate the transaction (act 203).

The system allows the medical services provider to select a network of contacts related to the medical services (act 204), such as contacts and targeted providers, including, but not limited to, primary care, internal medicine, family practice; endocrinology, rheumatology, pediatrics, chiropractics, physical therapy; physiatry, interventional pain, anesthesiology, cardiology, neurology, urgent care, behavioral health, dermatology, concierge medicine, medispas, ophthalmology, general surgery, orthopedics, bariatrics, vascular, plastic surgery, cardiac, neurosurgery, radiology; imaging centers, nutritionists, dentists, oral surgeons, cannabidiol (CBD), hemp dispensing and other medical services that one of skill in the art would understand.

The system may provide an invitation from the medical services provider, including, but not limited to, in the form of email, SMS, text, direct or indirect communication with a database or cloud services or other communication methods known to one of skilled in the art (act 205).

The system may provide a view of referral performance metrics, such as, but not limited to, referrals sent, received or networks associated with the medical services provider (act 206). Other reports may provide a holistic view of performance metrics, such as accounting or financial records or administrative records or communications transactions.

The system may allow the medical services provider to select a referral service, based on available possible medical services such as primary care, internal medicine, family practice; endocrinology, rheumatology, pediatrics, chiropractics, physical therapy; physiatry, interventional pain, anesthesiology, cardiology, neurology, urgent care, behavioral health, dermatology, concierge medicine, medispas, ophthalmology, general surgery, orthopedics, bariatrics, vascular, plastic surgery, cardiac, neurosurgery, radiology; imaging centers, nutritionists, dentists, oral surgeons, cannabidiol (CBD), hemp dispensing and other medical services that one of skill in the art would understand (act 207).

The system may allow the medical services provider to add medical services patients to the profile (act 208), including personal information, including insurance and medical information. Optionally, the system may allow a request for an administrator to send records related to a patient. Optionally, the system may allow the targeted provider or medical provider to contact each other via call-back mechanism.

The system may allow the medical services provider to send a referral notification to a targeted provider (act 209). Such notification may be in the form of telephonic, electronic, email, SMS, text, entry in a database, or other forms of communication known to one of skilled in the art.

The system may allow the medical services provider to receive a notification from the targeted provider (210). Such notification may be in the form of telephonic, electronic, email, SMS, text, entry in a database, or other forms of communication known to one of skilled in the art.

FIG. 2 also illustrates an example method that implements the disclosure. The system may use the elements of FIG. 1, including hardware elements disclosed in FIG. 1 that are adapted to perform the disclosure. The software is operable to perform acts to enable the embodiment. The software is operable to allow a medical services provider to create a profile (act 201). The medical services provider may create one or more credentials related to the profile and medical services (act 202). The profile may include name of the patient, email associated with the provider, photos for the profile, phone numbers associated with the provider, specialty associated with the provider, insurance information associated with the profile, location associated with the profile and administrator emails associated with the profile to facilitate the process.

The method allows a login process and graphical user interface to allow a login to the system, including username, email, and password. Other credentials may be possible. Other authentication, such as two factor or biometric authentication may be possible.

The disclosure allows the medical services provider to establish payment information, such as name, credit card or other possible payment information to facilitate the transaction (act 203).

The disclosed method allows the medical services provider to select a network of contacts related to the medical services (act 204), such as contacts and targeted providers, including, but not limited to, primary care, internal medicine, family practice; endocrinology, rheumatology, pediatrics, chiropractics, physical therapy; physiatry, interventional pain, anesthesiology, cardiology, neurology, urgent care, behavioral health, dermatology, concierge medicine, medispas, ophthalmology, general surgery, orthopedics, bariatrics, vascular, plastic surgery, cardiac, neurosurgery, radiology; imaging centers, nutritionists, dentists, oral surgeons, cannabidiol (CBD), hemp dispensing and other medical services that one of skill in the art would understand.

The disclosed method may provide an invitation from the medical services provider, including, but not limited to, in the form of email, SMS, text, direct or indirect communication with a database or cloud services or other communication methods known to one of skilled in the art (act 205).

The disclosed method may provide a view of referral performance metrics, such as, but not limited to, referrals sent, received or networks associated with the medical services provider (act 206). Other reports may provide a holistic view of performance metrics, such as accounting or financial records or administrative records or communications transactions.

The disclosed method may allow the medical services provider to select a referral service, based on available possible medical services such as primary care, internal medicine, family practice; endocrinology, rheumatology, pediatrics, chiropractics, physical therapy; physiatry, interventional pain, anesthesiology, cardiology, neurology, urgent care, behavioral health, dermatology, concierge medicine, medispas, ophthalmology, general surgery, orthopedics, bariatrics, vascular, plastic surgery, cardiac, neurosurgery, radiology; imaging centers, nutritionists, dentists, oral surgeons, cannabidiol (CBD), hemp dispensing and other medical services that one of skill in the art would understand (act 207).

The disclosed method may allow the medical services provider to add medical services patients to the profile (act 208), including personal information, including insurance and medical information. Optionally, the disclosed method may allow a request for an administrator to send records related to a patient. Optionally, the disclosed method may allow the targeted provider or medical provider to contact each other via call-back mechanism.

The disclosed method may allow the medical services provider to send a referral notification to a targeted provider (act 209). Such notification may be in the form of telephonic, electronic, email, SMS, text, entry in a database, or other forms of communication known to one of skilled in the art.

The disclosed method may allow the medical services provider to receive a notification from the targeted provider (210). Such notification may be in the form of telephonic, electronic, email, SMS, text, entry in a database, or other forms of communication known to one of skilled in the art.

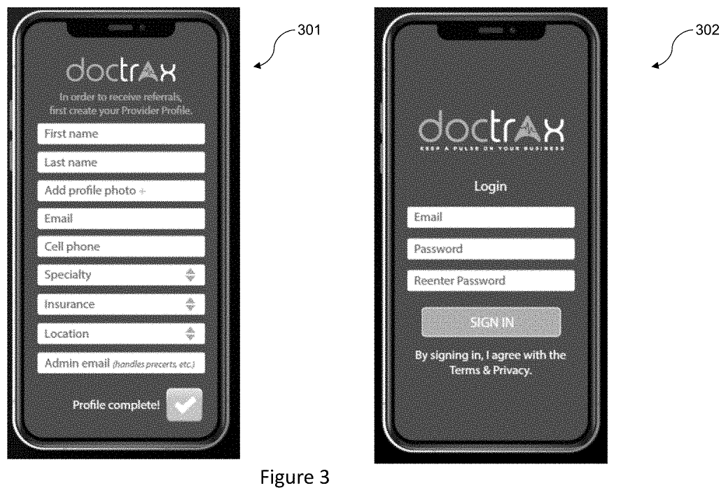

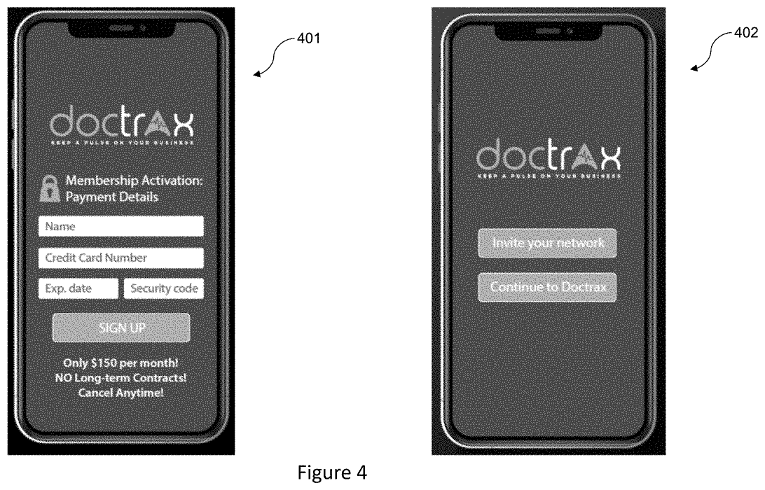

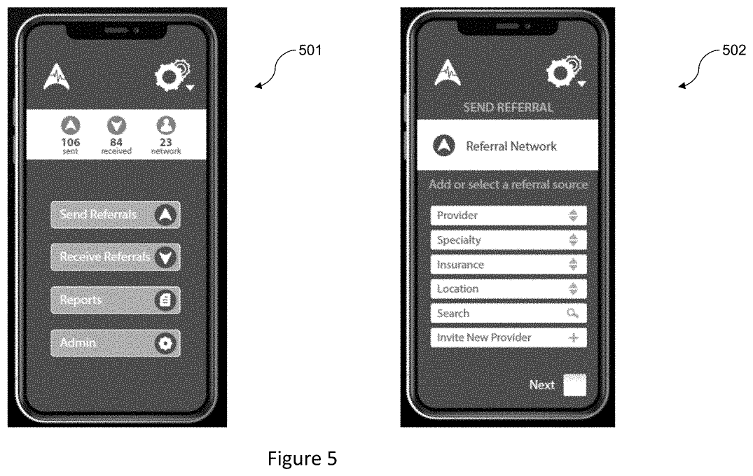

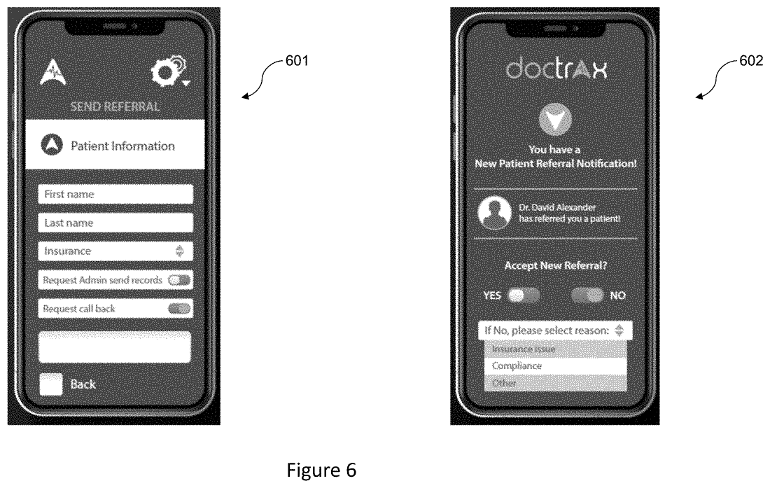

FIGS. 3-6 disclose another embodiment of the disclosure that discloses a computer-implemented method of for providing medical referral optimization on a graphical user interface (GUI) of a computer system.

The method is operable to allow the GUI to allow a user to perform acts to enable the embodiment. The software is operable to allow a medical services provider to create a profile (301). The medical services provider may create one or more credentials related to the profile and medical services. The profile may include name of the patient, email associated with the provider, photos for the profile, phone numbers associated with the provider, specialty associated with the provider, insurance information associated with the profile, location associated with the profile and administrator emails associated with the profile to facilitate the process.

The method is operable to allow the GUI to enable a user to allow a login to the system, including username, email, and password (302). Other credentials may be possible. Other authentication, such as two factor or biometric authentication may be possible.

The method is operable to let the GUI to allow a user to the medical services provider to establish payment information, such as name, credit card or other possible payment information to facilitate the transaction (401).

The disclosed method may provide an invitation from the medical services provider, including, but not limited to, in the form of email, SMS, text, direct or indirect communication with a database or cloud services or other communication methods known to one of skilled in the art (act 402).

The disclosed method may provide a view of referral performance metrics, such as, but not limited to, referrals sent, received or networks associated with the medical services provider (act 501). Other reports may provide a holistic view of performance metrics, such as accounting or financial records or administrative records or communications transactions.

The disclosed method allows the medical services provide, via the GUI, to select a network of contacts related to the medical services (act 501), such as contacts and targeted providers, including, but not limited to, primary care, internal medicine, family practice; endocrinology, rheumatology, pediatrics, chiropractics, physical therapy; physiatry, interventional pain, anesthesiology, cardiology, neurology, urgent care, behavioral health, dermatology, concierge medicine, medispas, ophthalmology, general surgery, orthopedics, bariatrics, vascular, plastic surgery, cardiac, neurosurgery, radiology; imaging centers, nutritionists, dentists, oral surgeons, cannabidiol (CBD), hemp dispensing and other medical services that one of skill in the art would understand.

The disclosed method may provide, via the GUI, an invitation from the medical services provider, including, but not limited to, in the form of email, SMS, text, direct or indirect communication with a database or cloud services or other communication methods known to one of skilled in the art.

The disclosed method may provide, via the GUI, a view of referral performance metrics (act, such as, but not limited to, referrals sent, received or networks associated with the medical services provider. Other reports may provide a holistic view of performance metrics, such as accounting or financial records or administrative records or communications transactions.

The disclosed method may provide, via the GUI, allow the medical services provider to select a referral service, based on available possible medical services such as primary care, internal medicine, family practice; endocrinology, rheumatology, pediatrics, chiropractics, physical therapy; physiatry, interventional pain, anesthesiology, cardiology, neurology, urgent care, behavioral health, dermatology, concierge medicine, medispas, ophthalmology, general surgery, orthopedics, bariatrics, vascular, plastic surgery, cardiac, neurosurgery, radiology; imaging centers, nutritionists, dentists, oral surgeons, cannabidiol (CBD), hemp dispensing and other medical services that one of skill in the art would understand.

The disclosed method may provide, via the GUI, the medical services provider to add medical services patients to the profile, including personal information, including insurance and medical information. Optionally, the method may allow a request for an administrator to send records related to a patient. Optionally, the method may allow the targeted provider or medical provider to contact each other via call-back mechanism.

The disclosed method may provide, via the GUI, the medical services provider to send a referral notification to a targeted provider. Such notification may be in the form of telephonic, electronic, email, SMS, text, entry in a database, or other forms of communication known to one of skilled in the art.

The disclosed method may provide, via the GUI, the medical services provider to receive a notification from the targeted provider. Such notification may be in the form of telephonic, electronic, email, SMS, text, entry in a database, or other forms of communication known to one of skilled in the art. The software is operable to perform acts to enable the embodiment. The method is operable to allow a medical services provider to create a profile. The medical services provider may create one or more credentials related to the profile and medical services. The profile may include name of the patient, email associated with the provider, photos for the profile, phone numbers associated with the provider, specialty associated with the provider, insurance information associated with the profile, location associated with the profile and administrator emails associated with the profile to facilitate the process.

The disclosed method may provide, via the GUI, a login process and graphical user interface to allow a login to the system, including username, email, and password. Other credentials may be possible. Other authentication, such as two factor or biometric authentication may be possible.

The disclosed method may provide, via the GUI, the medical services provider to establish payment information, such as name, credit card or other possible payment information to facilitate the transaction.

The disclosed method may provide, via the GUI, the medical services provider to select a network of contacts related to the medical services, such as contacts and targeted providers, including, but not limited to, primary care, internal medicine, family practice; endocrinology, rheumatology, pediatrics, chiropractics, physical therapy; physiatry, interventional pain, anesthesiology, cardiology, neurology, urgent care, behavioral health, dermatology, concierge medicine, medispas, ophthalmology, general surgery, orthopedics, bariatrics, vascular, plastic surgery, cardiac, neurosurgery, radiology; imaging centers, nutritionists, dentists, oral surgeons and other medical services that one of skill in the art would understand.

The disclosed method may provide, via the GUI, an invitation from the medical services provider, including, but not limited to, in the form of email, SMS, text, direct or indirect communication with a database or cloud services or other communication methods known to one of skilled in the art.

The disclosed method may provide, via the GUI, a view of referral performance metrics, such as, but not limited to, referrals sent, received or networks associated with the medical services provider. Other reports may provide a holistic view of performance metrics, such as accounting or financial records or administrative records or communications transactions.

The disclosed method may provide, via the GUI, the medical services provider to select a referral service, based on available possible medical services such as primary care, internal medicine, family practice; endocrinology, rheumatology, pediatrics, chiropractics, physical therapy; physiatry, interventional pain, anesthesiology, cardiology, neurology, urgent care, behavioral health, dermatology, concierge medicine, medispas, ophthalmology, general surgery, orthopedics, bariatrics, vascular, plastic surgery, cardiac, neurosurgery, radiology; imaging centers, nutritionists, dentists, oral surgeons and other medical services that one of skill in the art would understand.

The disclosed method may provide, via the GUI, the medical services provider to add medical services patients to the profile, including personal information, including insurance and medical information. Optionally, the method may allow a request for an administrator to send records related to a patient. Optionally, the method may allow the targeted provider or medical provider to contact each other via call-back mechanism.

The disclosed method may provide, via the GUI, the medical services provider to send a referral notification to a targeted provider. Such notification may be in the form of telephonic, electronic, email, SMS, text, entry in a database, or other forms of communication known to one of skilled in the art.

The disclosed method may provide, via the GUI, the medical services provider to receive a notification from the targeted provider. Such notification may be in the form of telephonic, electronic, email, SMS, text, entry in a database, or other forms of communication known to one of skilled in the art. The disclosed method may provide an invitation from the medical services provider, including, but not limited to, in the form of email, SMS, text, direct or indirect communication with a database or cloud services or other communication methods known to one of skilled in the art.

The disclosed method may provide, via the GUI, a view of referral performance metrics, such as, but not limited to, referrals sent, received or networks associated with the medical services provider. Other reports may provide a holistic view of performance metrics, such as accounting or financial records or administrative records or communications transactions.

The disclosed method may provide, via the GUI, the medical services provider to select a referral service, based on available possible medical services such as primary care, internal medicine, family practice; endocrinology, rheumatology, pediatrics, chiropractics, physical therapy; physiatry, interventional pain, anesthesiology, cardiology, neurology, urgent care, behavioral health, dermatology, concierge medicine, medispas, ophthalmology, general surgery, orthopedics, bariatrics, vascular, plastic surgery, cardiac, neurosurgery, radiology; imaging centers, nutritionists, dentists, oral surgeons, cannabidiol (CBD), hemp dispensing and other medical services that one of skill in the art would understand.

The disclosed method may provide, via the GUI, the medical services provider to add medical services patients to the profile, including personal information, including insurance and medical information. Optionally, the system may allow a request for an administrator to send records related to a patient. Optionally, the system may allow the targeted provider or medical provider to contact each other via call-back mechanism.

The disclosed method may provide, via the GUI, allow the medical services provider to send a referral notification to a targeted provider. Such notification may be in the form of telephonic, electronic, email, SMS, text, entry in a database, or other forms of communication known to one of skilled in the art.

The method may allow the medical services provider to receive a notification from the targeted provider. Such notification may be in the form of telephonic, electronic, email, SMS, text, entry in a database, or other forms of communication known to one of skilled in the art.

The sequence diagrams of FIGS. 1-6 may be encoded in a signal bearing medium, a computer readable medium such as a memory, programmed within a device such as one or more integrated circuits, or processed by a controller or a computer. If the methods are performed by software, the software may reside in a memory resident to or interfaced to a receiver, a transmitter, a communication interface, or any other type of non-volatile or volatile memory interfaced or resident to the data transmission system. The memory may include an ordered listing of executable instructions for implementing logical functions. A logical function may be implemented through digital circuitry, through source code, through analog circuitry, or through an analog source such through an analog electrical, audio, or video signal. The software may be embodied in any computer-readable or signal-bearing medium, for use by, or in connection with an instruction executable system, apparatus, or device. Such a system may include a computer-based system, a processor-containing system, or another system that may selectively fetch instructions from an instruction executable system, apparatus, or device that also may execute instructions.

A “computer-readable medium,” “machine-readable medium,” “propagated-signal” medium, and/or “signal-bearing medium” may include any means that contains, stores, communicates, propagates, or transports software for use by or in connection with an instruction executable system, apparatus, or device. The machine-readable medium may selectively be, but not limited to, an electronic, magnetic, optical, electromagnetic, infrared, or semiconductor system, apparatus, device, or propagation medium. A non-exhaustive list of examples of a machine-readable medium would include: an electrical connection “electronic” having one or more wires, a portable magnetic or optical disk, a volatile memory such as a Random Access Memory “RAM” (electronic), a Read-Only Memory “ROM” (electronic), an Erasable Programmable Read-Only Memory (EPROM or Flash memory) (electronic), or an optical fiber (optical). A machine-readable medium also may include a tangible medium upon which software is printed, as the software may be electronically stored as an image or in another format (e.g., through an optical scan), then compiled, and/or interpreted or otherwise processed. The processed medium may then be stored in a computer and/or machine memory.

While various embodiments of the invention have been described, it will be apparent to those of ordinary skill in the art that many more embodiments and implementations are possible within the scope of the invention. Accordingly, the invention is not to be restricted except in light of the attached claims and their equivalents.

I claim:

1. A system for medical referral optimization, comprising:
a processor;
a memory device coupled to the processor; and
software operable on the processor and memory device for:
allowing a medical services provider to create a profile;
allowing the medical services provider to create one or more credentials related to the profile and medical services;
allowing the medical services provider to establish payment information;
allowing the medical services provider to select a network of contacts related to the medical services;
providing an invitation from the medical services provider;
providing a view of referral performance metrics;
allowing the medical services provider to select a referral service;
allowing the medical services provider to add medical services patients to the profile;
sending a referral notification to a targeted provider;
receiving a notification from the targeted provider.

2. The system of claim 1 where the credentials comprise a username associated with the medical services provider.

3. The system of claim 1 where the credentials comprise a password associated with the medical services provider.

4. The system of claim 1 where the performance metrics comprise data associated with a number of transactions between the medical services provider and the targeted provider.

5. The system of claim 1 where the medical services provider transmits a message to an external administrative part for insurance verification and scheduling.

6. The system of claim 1 where the medical services provider accepts or rejects the notification from the targeted provider.

7. The system of claim 1 where a second notification is transmitted to the targeted provider.

8. The system of claim 1 where the system proactively provides information to the medical services providers and patients on the insurance plans that targeted provider accepts.

9. A method for medical referral optimization, comprising:
allowing a medical services provider to create a profile;
allowing the medical services provider to create one or more credentials related to the profile and medical services;
allowing the medical services provider to establish payment information;
allowing the medical services provider to select a network of contacts related to the medical services;
providing an invitation from the medical services provider;
providing a view of referral performance metrics;
allowing the medical services provider to select a referral service;
allowing the provider to add medical services patients to the profile;
sending a referral notification to a targeted provider;
receiving a notification from the targeted provider.

10. The method of claim 8 where the credentials comprise a username associated with the medical services provider.

11. The method of claim 8 where the credentials comprise a password associated with the medical services provider.

12. The method of claim 8 where the performance metrics comprise data associated with a number of transactions between the medical services provider and the targeted provider.

13. The method of claim 8 where the medical services provider transmits a message to an external administrative part for insurance verification and scheduling.

14. The method of claim 8 where the medical services provider accepts or rejects the notification from the targeted provider.

15. The method of claim 8 where a second notification is transmitted to the targeted provider.

16. The method of claim 8 where the system proactively provides information to the medical services providers and patients on the insurance plans that targeted provider accepts

17. A method of for providing medical referral optimization on a graphical user interface (GUI) of a computer system, the method comprising:
allowing a medical services provider to create a profile;
allowing the medical services provider to create one or more credentials related to the profile and medical services;
allowing the medical services provider to establish payment information;
allowing the medical services provider to select a network of contacts related to the medical services;
providing an invitation from the medical services provider;
providing a view of referral performance metrics;
allowing the medical services provider to select a referral service;
allowing the provider to add medical services patients to the profile;
sending a referral notification to a targeted provider;
receiving a notification from the targeted provider.

18. The method of claim 15 where the GUI allows the medical services provider to enter credentials comprise a username associated with the medical services provider.

19. The method of claim 15 where the GUI allows the medical services provider to enter the credentials which comprise a password associated with the medical services provider.

20. The method of claim 15 where the GUI displays the performance metrics which comprise data associated with a number of transactions between the medical services provider and the targeted provider.

21. The method of claim 15 where the GUI allows the medical services provider to transmit a message to an external administrative part for insurance verification and scheduling.

22. The method of claim 15 where the GUI allows the medical services provider to accept or reject the notification from the targeted provider.

23. The method of claim 22 where the GUI proactively provides information to the medical services providers and patients on the insurance plans that targeted provider accepts
